(19) United States
(12) Patent Application Publication
(10) Pub. No.: US 2024/0065759 A1
(43) Pub. Date: Feb. 29, 2024

(54) ELECTROSURGICAL ACCESS SHEATH

(71) Applicant: Broncus Medical Inc., San Jose, CA (US)

(72) Inventors: Eric Gwerder, San Jose, CA (US); Thomas Keast, Sunnyvale, CA (US); Henky Wibowo, San Jose, CA (US)

(73) Assignee: Broncus Medical Inc., San Jose, CA (US)

(21) Appl. No.: 18/502,289

(22) Filed: Nov. 6, 2023

Related U.S. Application Data
(63) Continuation of application No. 16/496,425, filed on Sep. 21, 2019, now Pat. No. 11,832,877.
(60) Provisional application No. 62/480,539, filed on Apr. 3, 2017.

(51) Int. Cl. A61B 18/14; A61B 90/00

(52) U.S. Cl. CPC A61B 18/1492; A61B 90/37; A61B 2018/00077; A61B 2090/376

(57) ABSTRACT

An electrosurgical access catheter for assisting a physician perform an ancillary surgical procedure on a region of interest (ROI) in a patient has a tubular shaped elongate shaft and a ring-shaped active electrode located at the distal end. A passageway extends from the proximal section to the distal end of the shaft, and through the ring-shaped active electrode. The proximal section of the elongate shaft is detachably coupled to an electrosurgical controller such that the ring-shaped active electrode is operable with the electrosurgical controller and a dispersive electrode to electrosurgically make a circular-shaped incision through a tissue wall, carving a cylindrical-shaped tissue plug as the ring-shaped active electrode is advanced through the wall. The access sheath is advanced through the surgically created opening and without further dilation of the opening. The invention has particular applicability to the lung, and minimally invasive bronchoscopic procedures. Related methods are also described.

### CROSS-REFERENCE TO RELATED APPLICATIONS

This application is a continuation application of non-provisional application Ser. No. 16/496,425, filed Sep. 21, 2019, which is a national phase filing under 35 USC Section 371 of international patent application no. PCT/US18/25756, filed Apr. 2, 2018, which claims priority to provisional patent application No. 62/480,539, filed Apr. 3, 2017, each of which is entitled “ELECTROSURGICAL ACCESS SHEATH” and each which is incorporated herein by reference for all purposes.

### BACKGROUND OF THE INVENTION

The invention relates to surgical instruments, and more particularly, to bronchoscopic access catheters having electrosurgical functionality to cut lung tissue.

In various medical procedures, access catheters are advanced through the natural lumens of the body and to a target tissue site where a medical procedure is carried out. The access catheter serves to prevent collateral damage to tissue as surgical tools are advanced from outside the body to the target site, and in the case of tumor biopsy and cancer treatments, the access catheter potentially prevents spreading of cancer cells as the biopsy sample is collected and withdrawn from the body.

The access catheters must be large enough to accommodate the tools which are to be passed through the access catheter. The relatively large size of the access catheter necessary to accommodate the tools, however, prohibits the access catheter from reaching smaller more remote target areas without damaging the tissue. Consequently, dilation and expansion of tissues is required in order to advance the access catheter into position. Additional tools and instruments are required to safely dilate tissue spaces, and to advance the access catheter into position. Undesirably, in some cases, multiple steps and tool exchanges may be required involving inflatable balloons, and dilating instruments.

Notwithstanding the above, an apparatus and related method that overcomes the above mentioned challenges is desirable.

### SUMMARY OF THE INVENTION

An electrosurgical access sheath system and method enables a physician to reach target regions of interest exterior to the airways in the lung of a patient.

In embodiments, the access sheath creates large incisions using radio frequency (RF) energy. After an opening is created using the RF energy, the access sheath may be advanced through the opening without dilating the opening. In embodiments, the radio frequency energy is monopolar-based based.

In embodiments, the distal working section of the access sheath through the opening has a constant diameter.

In embodiments, subsequent to placing the access sheath in position, various medical procedures are performed through the access sheath including diagnostic and treatment procedures. In embodiments, tissue biopsy or electrosurgical ablation is performed on suspect growths.

Still other descriptions, objects and advantages of the present invention will become apparent from the detailed description to follow, together with the accompanying drawings.

### BRIEF DESCRIPTION OF THE DRAWINGS

FIGS. 1A-1C sequentially illustrate a bronchoscopic-based technique for advancing an access sheath from within a lung airway to a region of interest located outside of the airway in the lung parenchyma.

FIG. 2 is a representative fluoroscopic image corresponding to FIG. 1C showing the access sheath location.

FIGS. 3, 4 illustrate carrying out a biopsy and ablation procedure on the region of interest through the access sheath, respectively.

FIGS. 5A, 5B are partial perspective views of an electrosurgical stylet in a retracted and extended state respectively.

FIG. 6 is an illustration of a monopolar-based electrosurgical system.

FIGS. 7A, 7B are sequential illustrations of an electrosurgical stylet being positioned and subsequently cutting the target tissue.

FIG. 8 is a partial perspective view of another electrosurgical instrument including a loop shaped electrode.

FIG. 9 is a partial perspective view of an access sheath including a ring shaped cutting electrode.

FIG. 10 is an illustration of the access sheath represented in FIG. 9 electrosurgically cutting tissue.

FIG. 11A is an illustration of several circular-shaped incisions created with an access sheath as shown in FIG. 9.

FIG. 11B is an illustration of a tissue plug captured from the access sheath represented in FIG. 9.

FIG. 12 is a side view of an electrosurgical access sheath.

FIGS. 13, 14 illustrate sequentially steps to manufacture an electrosurgical access sheath.

FIGS. 15A, 15B are partial side and end views respectively of another electrosurgical access sheath.

FIG. 15C is a partial perspective view of the electrosurgical access sheath shown in FIGS. 15A, 15B.

FIG. 16 is an illustration of another electrosurgical access sheath.

FIGS. 17A, 17B are illustrations of another electrosurgical access sheath having a retracted and extended state respectively.

FIGS. 18A, 18B are illustrations of another electrosurgical access sheath having a cutting annulus in an exposed and neutralized state respectively.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

The following patents and applications are incorporated herein by reference in their entirety: U.S. Pat. Nos. 6,692,494, 8,709,034, 8,784,400, 7,393,330, and 9,265,468 and US Patent Publication No. 2013/0123638.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

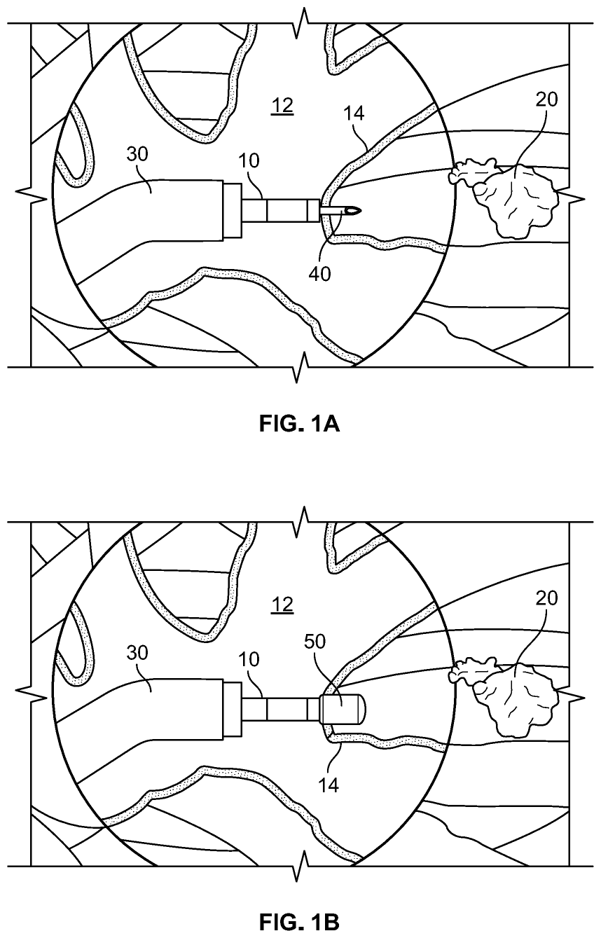

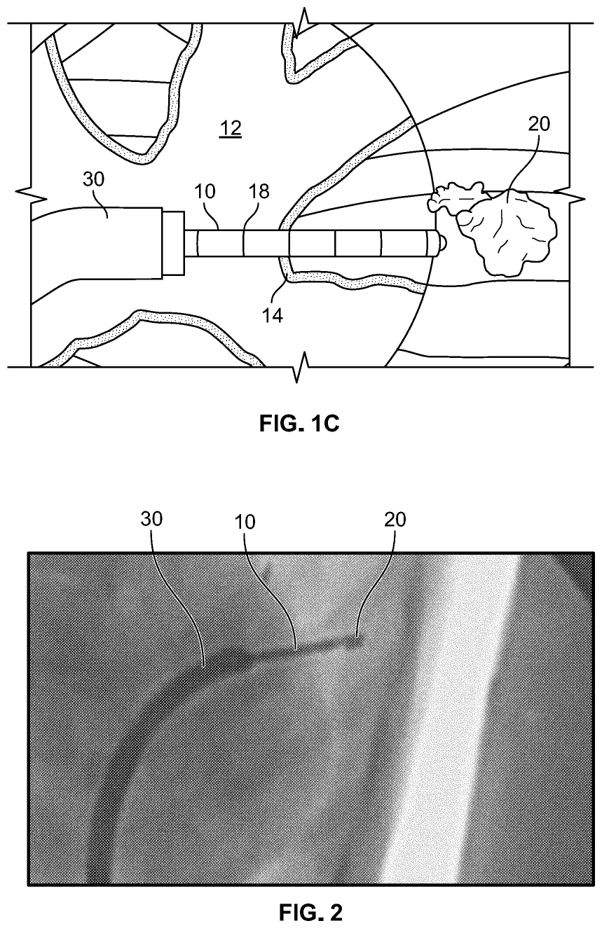

FIGS. 1-3 sequentially illustrate a bronchoscopic-based technique for performing a medical procedure on a region of interest 20 located outside of the airway 12 in the lung parenchyma. The region of interest 20 may be spaced a distance from the airway from 3-40 mm, and in some cases, 10-15 mm. Examples of medical procedures include diagnostic and treatment type procedures including without limitation biopsy, ablation, seed placement, and local chemical agent delivery.

With reference first to FIG. 1A, a bronchoscope 30 is shown within airway 12, and in close proximity to airway wall 14. Access sheath 10 extends from the distal end of bronchoscope 30. The access sheath is close to, or perhaps in contact with, the airway wall 14. Needle 40 is advanced from the distal end of the access sheath 10 in the direction of the region of interest (ROI) 20, creating a hole through the airway wall 14.

FIG. 1B shows dilation member 50 inserted through pre-existing hole in the airway wall 14. Dilation member 50 is enlarged to expand the hole. In some instances, the hole is expanded by a factor of 2 to 4 (in some embodiments, about a factor of 3) more than the hole created by the needle. Dilation member 58 may be an inflatable balloon, expandable member, or another type of dilating member such as a tapered dilator.

FIG. 1C shows access sheath 10 advanced through the enlarged/dilated hole in the airway wall 14, and to the region of interest 20. Marker bands 18 are shown on the sheath to provide a visual indication of the depth of the sheath, or motion of the sheath. Marker bands may be ink, and evenly spaced axially along the sheath.

The region of interest is shown as a tissue mass such as a tumor. However, the region of interest may include others tissues, structures, or implants for which a procedure is desirable. It should be noted, however, that once the access catheter 10 is advanced beyond the airway 12, it is outside the view of the bronchoscope.

FIG. 2 shows a two dimensional fluoroscopic image view of the access sheath. Access sheath 10 is extending from bronchoscope 30, and towards the region of interest 20. The airways are not visible under the fluoroscopic view. The fluoroscopic view serves to track the access sheath and confirm the location of the instruments once the tools are outside of the airways, and outside the view of the bronchoscope.

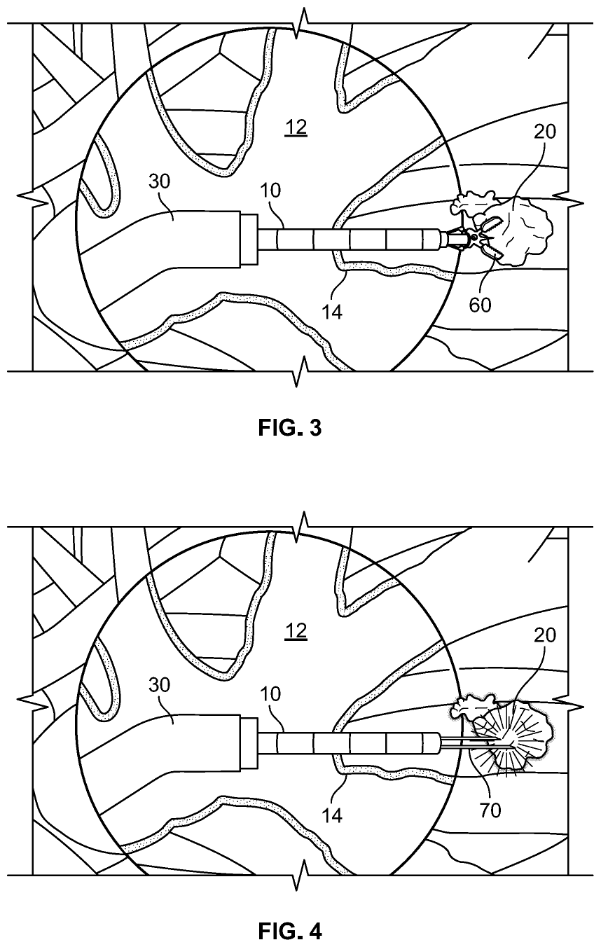

FIG. 3 shows a biopsy forceps 60 extending from the distal end of the access sheath 10. The biopsy forceps 60 obtains a sample specimen from the region of interest.

FIG. 4 shows ablation needles 70 extending from the distal end of the access sheath 10, and into the region of interest 20. The ablation needles 70 are activated to destroy the region of interest. Although forceps and ablation needles are shown, a wide range of tools may be delivered through the access sheath to carry out different types of medical procedures.

Electrosurgical Guide Wire

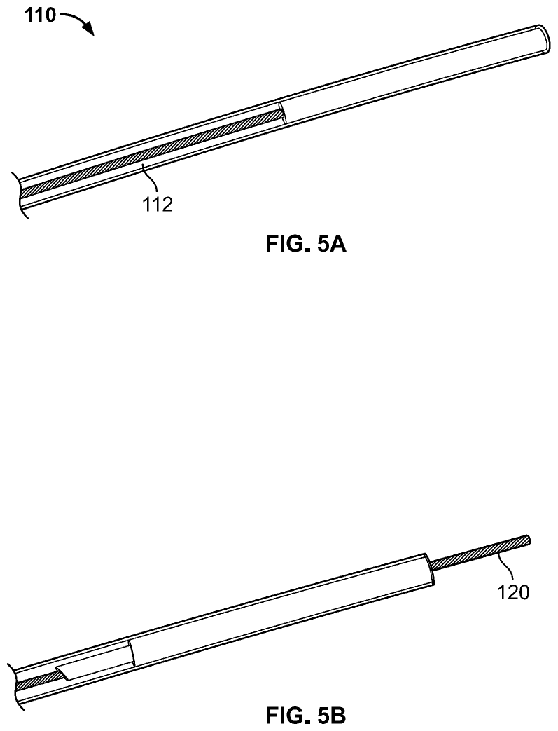

FIGS. 5A, 5B are partial perspective views of an electrosurgical stylet or guide member 110 in a retracted and extended state respectively.

The stylet 110 includes an outer cover 112, and inner member 120 axially movable relative to the cover. The distal section of the inner member terminates in an active electrode 120. The active electrode may be a wire with a blunted edge. In some embodiments, the wire may have a sharp tip whether beveled or otherwise. In other embodiments, the wire may have a rounded atraumatic ball or spherical shaped tip.

While in the retracted state shown in FIG. 5A, the stylet 100 is advanced through the working lumen of the bronchoscope, and into airway lumens. The outer cover 112 prevents the tip of the wire-shaped active electrode 120 from stabbing or damaging the bronchoscope or airway lumens.

The active electrode 120 is in electrical communication with a wire conductor (not shown) extending proximally, and coupled to an electrosurgical controller (not shown). Additionally, in embodiments, the active electrode 120 itself may be in the form of an elongate wire, and extend proximally to a detachable connector for coupling to the controller. Detachable connectors include, without limitation, removable plug-in type connectors. However, other types of connectors may be incorporated into the systems described herein to couple the guide member and active electrode to the controller.

The materials for the inner member 120 may vary widely. In embodiments, the inner member is formed of a metal or alloy. Examples of materials for the inner member include, without limitation, electrical conducting materials such as copper, stainless steel, Nitinol, and titanium.

The materials for the cover 112 may also vary. Examples of materials for the outer cover include, without limitation, extruded polymeric tubing formed of PEBAX, FEP, PTFE, PE, and Nylon.

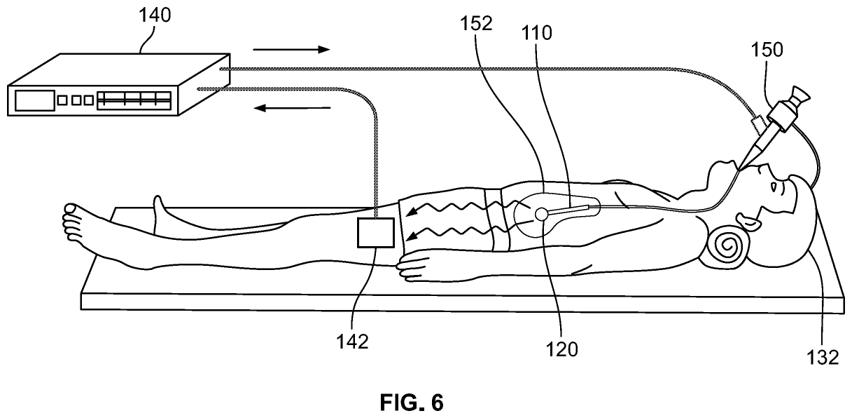

With reference to FIG. 6, the guide member 110 is advanced from a bronchoscope 150 to a target position along an airway in the lung 152 of the patient 132. A controller 140 is shown electrically connected to the active electrode 120 and a dispersive electrode 142 affixed to the skin of the patient.

In embodiments, an alternating current (e.g., a RF current waveform) is applied to the circuit causing the active electrode to cut the target tissue in close vicinity of the active electrode. In embodiments, a sinusoidal voltage at a frequency of 400 kHz±100 kHz is applied. Power setting may vary from 5-300 watts. Timing for cutting may also vary. In embodiments, the energy is activated for the time required to cut (typically between 0.1 and 5 seconds; however, depending on the anatomy, tissue moisture content, salinity, and type (airway, parenchyma, tumor, etc.), cut times will vary. An example of a controller is the VIO 300D, manufactured by Erbe USA, Inc., Marietta, Georgia. Examples of electrosurgical controllers for use with the electrosurgical instruments of the present invention are described in the literature including, without limitation, U.S. Pat. No. 5,599,344, US Patent Publication Nos. 20080249523, 20110190755, each of which is incorporated herein.

In addition to cutting, in embodiments, the controllers are operable to cauterize by application of a different energy setting typically referred to as the controller's coagulation mode. Providing a coagulation mode can add safety to the procedure by sealing small vessels that may be severed during a cutting procedure. Without intending to being bound to theory, coagulation is generally associated with a lower temperatures, modulated current, and higher voltages (up to 10,000 V peak to peak) to cause relatively deep tissue destruction than the cutting mode. Additionally, in embodiments the electrosurgical device is used a standalone product for performing electrocautery.

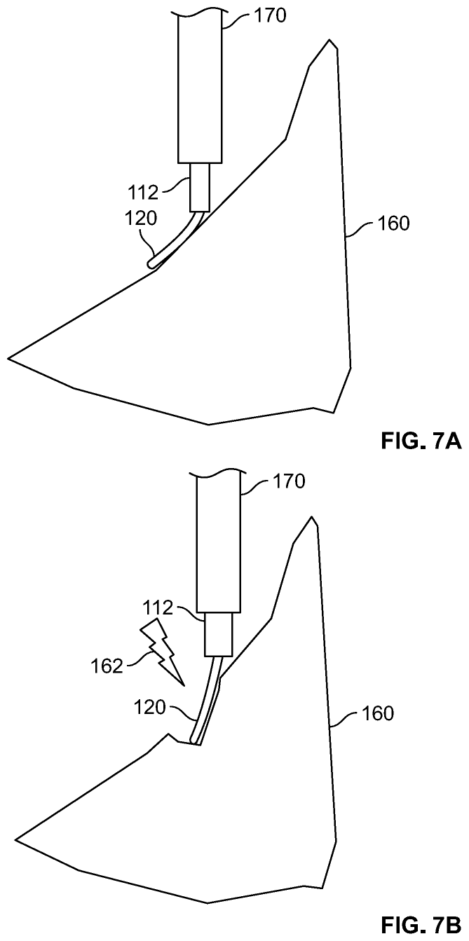

FIG. 7A illustrates the electrosurgical guide member described in FIGS. 5A, 5B, 6 cutting tissue 160. The active electrode 120 is advanced from the cover 112 and towards the airway wall. The active electrode 120 is further advanced against the tissue surface, conforming to the tissue surface. The active electrode is shown curving along the surface of the tissue such that a lateral surface of the electrode 120 is in contact with the tissue 160.

FIG. 7B shows application of energy 162 to the active electrode, cutting the tissue 160.

In the embodiment shown in FIG. 7B, the active electrode includes several cutting regions including a lateral circumferential region and the front end/edge. The length of lateral cutting area ranges from 1−20 mm. The tip has a diameter of 0.5 mm.

The multiple electrode cutting regions shown in the embodiment of FIG. 7B enable the physician to make larger holes than would be created if only the front edge region was active. Hole diameters corresponding to the embodiment shown in FIG. 7B are dependent upon the exposed electrode length 1 to 40 times larger than a hole created using only a wire tip, or front-only ablation.

After a hole, opening, or channel is created through the airway wall tissue 160, the wire 120 may be retracted into the guide member 112, and the guide member assembly exchanged for an access sheath 170. The guide sheath is advanced through the hole without the need to dilate the hole.

In alternative embodiments, the guide member assembly 110 is advanced beyond the airway wall and through parenchyma tissue to the ROI. Fluoroscopic guidance may be used to track the location of the guide member assembly outside of the airways.

After the guide member assembly reaches the target location, an access sheath 170 may be advanced over the guide member, through the pre-cut hole, and to the ROI (not shown). Advantageously, the access sheath 170 may be advanced to the ROI without the need for dilating the hole. Various medical procedures may be performed through the access sheath 170 as described herein.

Electrosurgical Guide Loop Electrode

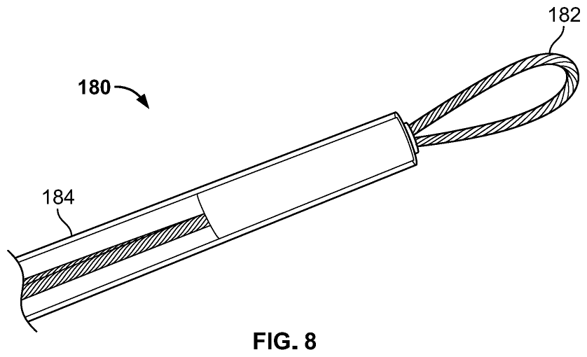

FIG. 8 is a partial perspective view of another electrosurgical guide instrument 180 including a loop shaped electrode 182. The loop electrode is shown protruding from an outer cover 184. The loop 182 and cover 184 may be made of similar materials to that used for the wire electrode member 120 and cover 112 shown and described in FIGS. 5A, 5B, respectively.

The width and length of the loop 182 preferably ranges from 1-3 mm and 2-10 mm, respectfully. Consequently, when activated, the loop electrode can make a relatively large cut through the airway wall. Additionally, because the loop electrode 182 naturally has a curved atraumatic shape, it may be advanced through the working lumen of a bronchoscope and airways without inadvertently piecing or damaging the lumen walls.

Once a hole has been created, the access sheaths described herein may be advanced through the surgically created hole to the ROI. The access sheath may be advanced into position over the loop guide instrument 180, or pushed into position with the loop instrument 180 previously removed. Using either technique, the access sheath is advanced through the hole without further dilation or enlargement of the hole.

Additionally, in embodiments, the inner wire 120 or loop members 182 may be configured to be actuatable from the proximal end of the device. Indeed, any of the active electrodes for cutting described herein may be configured to, or linked to, a handle or actuator assembly to extend and retract the active electrode from the sheaths. Various actuator mechanisms for use with the piercing members and electrodes are described herein.

Electrosurgical Access Sheath

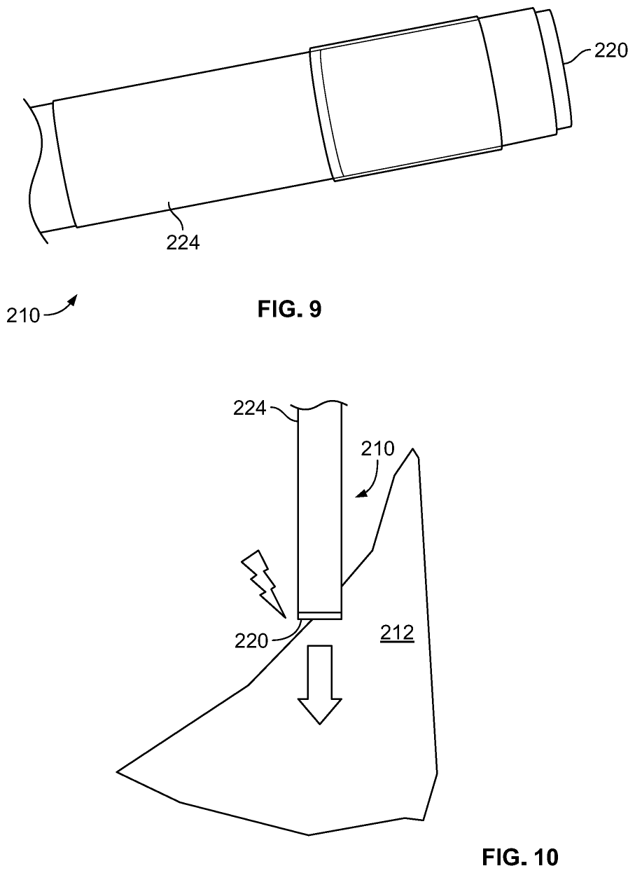

FIG. 9 is a partial perspective view of the distal section of another access sheath 210 including a ring shaped cutting electrode 220. The ring shaped electrode 220 protrudes from a tubular shaped body 224. An electrical conductor, braid, is in electrical contact with the electrode and extends proximally through the tubular body to the proximal section where the conductor is coupled to a controller.

FIG. 10 illustrates the access sheath 210 cutting an opening through a tissue 212 when activated as described herein. The access sheath 210 is activated and advanced through the tissue, for example, the airway wall, creating a port through which the access sheath may be advanced.

Additionally, the access sheath is advanced through the hole without further dilation or enlargement of the hole. In embodiments, the outer diameter of the access sheath is approximately the same as the ring electrode. The outer diameter of the access sheath may range from 2 to 3 mm. The sheath diameter may be constant.

As described herein, the sheath and electrode are sized to accommodate procedural tools therethrough. In embodiments, the inner diameter of the electrode and sheath ranges from 1.8-2.8 mm. The electrode and sheath may be made of materials as described above in connection with the electrode 120 and outer cover 112 of FIGS. 5A, 5B. The electrode may be made of a platinum, iridium, stainless steel, tungsten, titanium, and other electrically conducting and x-ray visible materials for assisting the physician locate and track the instrument during a procedure. Further details of manufacturing the sheath 210 are described herein.

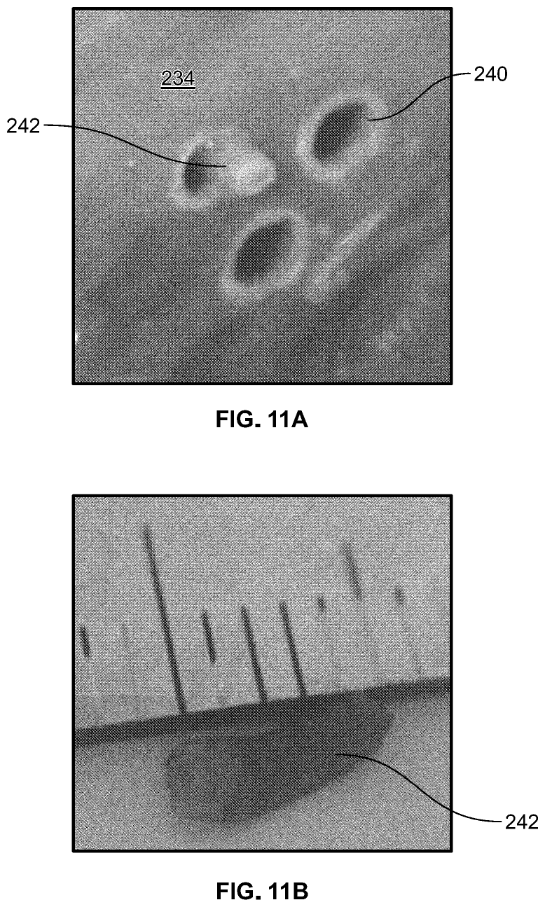

FIG. 11A is an illustration of several holes 240 in a tissue 234 made by the access sheath 210 described in FIG. 9. The tissue 234 is bovine liver. Power setting for cutting the holes ranged from 30 to 100 watts. The holes 240 are clearly defined, large (about 2 mm in diameter), and result in procuring or carving a tissue plug 242. The length of plug 242 with reference to FIG. 11B is about 5 mm of contiguous tissue.

Large sample biopsies are anticipated using the access sheath described herein. Evidence from sampling bovine liver described herein shows sample sizes as large as 20 mm long and 2 mm in diameter. In embodiments, a method and apparatus includes obtaining the tissue plugs described herein for use in minimally invasive mediastinoscopy where large sample size is highly desired. Additionally, biopsy sampling can be performed at other lymphatic sites and/or tumors.

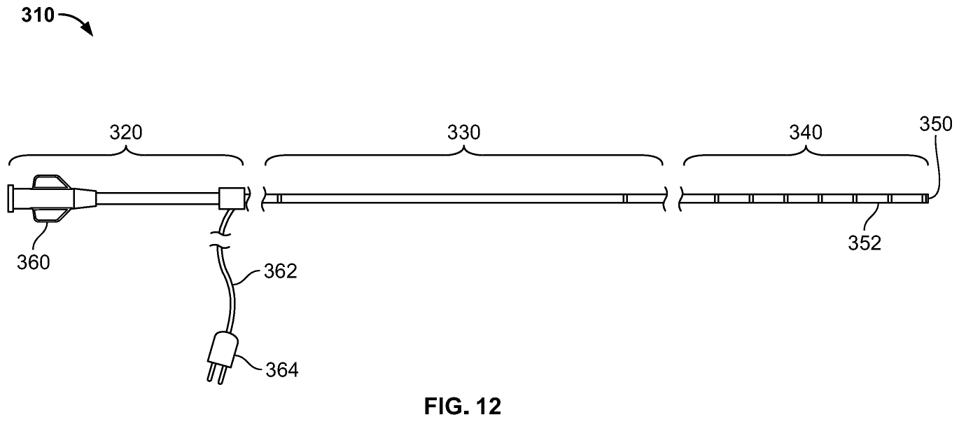

FIG. 12 is a side view of an entire electrosurgical access sheath 310 in accordance with one embodiment of the invention. It includes a flexible tubular shaft having a proximal section 320, intermediate section 330, and distal section 340. As described above the tubular shaft is sized for various tools to be passed therethrough such as, without limitation, biopsy forceps, aspiration needles, electrosurgical and cryo-ablation catheters, snares, etc. The inner diameter of the tubular shaft may range from 1.8 to 2.8 mm, and preferably is about 2.0 mm. The outer diameter is less than 3 mm. In the embodiment shown in FIG. 12, the shaft intermediate and distal sections have a constant outer diameter.

Radiopaque markers 352 are shown in the form of bands evenly spaced along the distal section of the sheath. Bands 352 may be formed of various radiopaque materials including, for example, platinum, iridium, and other fluoroscopically opaque materials.

The proximal section 320 is shown having a hub 360 such as a Luer connector. Certain tools as described above may be advanced through the sheath, and conveniently connected with hub 360.

Electrical cable 362 is shown extending from the proximal section. Electrical cable terminates in a connector which may be detachably engaged with controller not shown. As will be described further herein, in embodiments, electrical cable 362 includes electrical conducting lines which are in electrical communication with the distal ring electrode 350 of the sheath.

The total length of the access sheath 310 may vary. In embodiments, the length from the hub 360 to the ring electrode 350 is about 35-45 inches preferably about 35-40 inches. In embodiments, the access sheath 310 has a total length suitable to be inserted through the working lumen of a bronchoscope in a patient, beyond the end of the bronchoscope tip, and to a remote or peripherally-located tissue growth or nodule. In embodiments, the nodules and ROIs may be located in the parenchyma tissue outside of the airways 3 to 100 mm from the end of the scope, or from the wall of the airway.

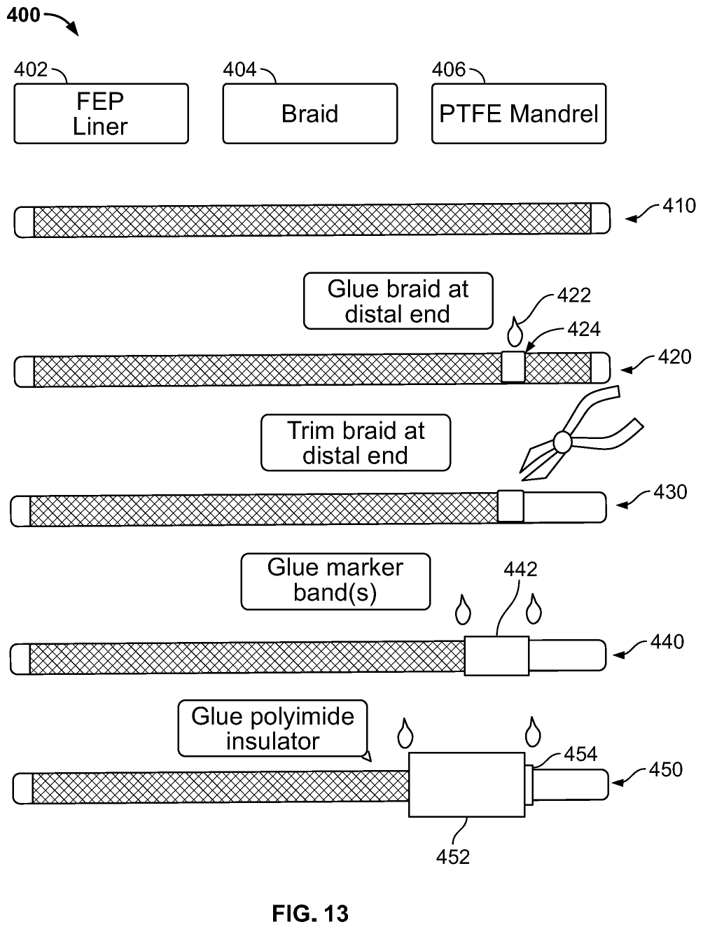

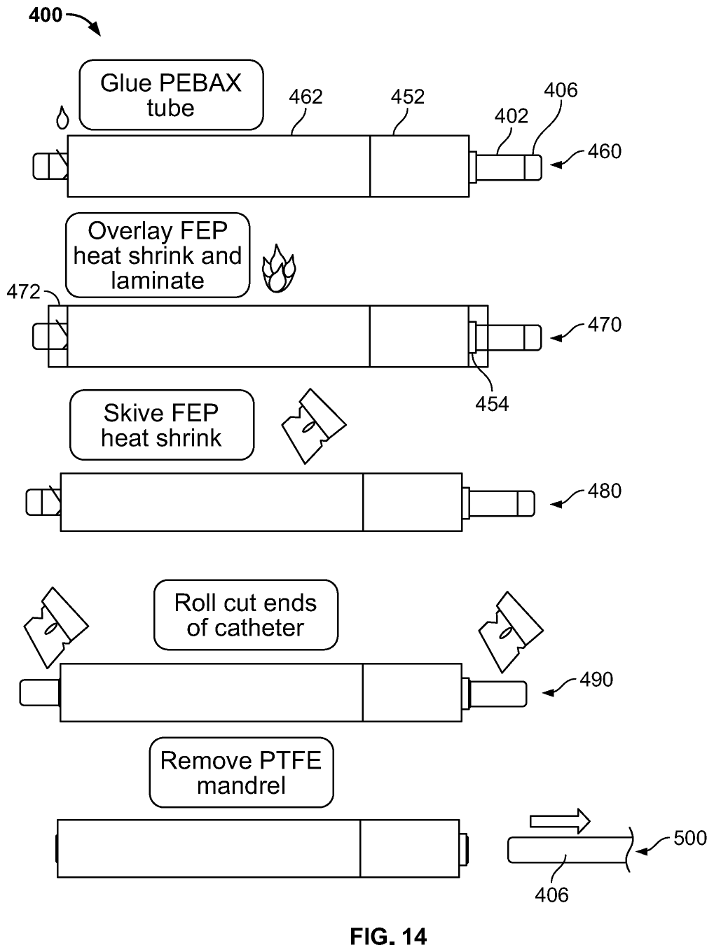

FIGS. 13, 14 illustrate a process 400 to manufacture an electrosurgical access sheath in accordance with embodiments of the invention.

Initially, with reference to step 410, a fluorinated ethylene-propylene (FEP) liner 402 is shown surrounding a polytetrafluoroethylene (PTFE) mandrel having an outer diameter of about 2 mm. A braid 404 is shown surrounding the liner 402.

The pitch of the braid may range from 20 to 100 PPI. Additionally, the flexibility of the sheath may be varied by changes to the braid pitch (e.g. tight or loose pitch, the braid to coil transition(s), and the thickness of the braiding material). A preferred braid configuration would be a 16 spool woven construct of 0.001″×0.003″ flat wire, from stainless steel, with a pick count of 48 PPI. Additionally, in embodiments, the braid is made of an electrical conducting material and serves as the electrical conductor between the controller and the active electrode.

Step 420 illustrates bonding the braid 404 to the liner 402 at the distal end 424. Bonding may be carried out, e.g., using various adhesives 422.

Step 430 illustrates trimming the braid at the distal end of the sheath.

Step 440 illustrates fastening an elongated tubular metal electrode 442 to the braid 404 and liner 406. The tubular metal electrode is shown being glued in place.

Step 450 illustrates covering the proximal region of the metal electrode 442 with an insulator 452, thereby defining a ring-shaped electrode 454.

The insulator serves to protect the active electrode from damaging other parts of the sheath in the vicinity of the active electrode. Exemplary materials for the insulator include without limitation polyimide, fluoropolymer, PEEK, aluminum oxide (alumina), and parylene.

Step 460 illustrates a polymer tubing 462 bonded over the braid/liner. Exemplary materials for the polymer tubing may be PEBAX. The polymer tubing 462 may be bonded with, e.g., glue.

Step 470 illustrates an outer layer 472 of FEP surrounding the polymer tubing 462, insulator 452, and ring electrode 454. The outer layer of FEP is heat-shrunk in place.

Step 480 illustrates trimming (e.g., skive off) the FEP layer, to expose the laminated polymer tubing 462.

Step 490 illustrates trimming (e.g., roll cutting) the inner FEP liner 402 and excess braid.

Step 500 shows removing the mandrel 406, and completing the build of the shaft of the electrosurgical access sheath.

Additionally, an electrical cable (e.g., the cable 362 shown in FIG. 12) is installed to the access sheath. In embodiments, the proximal section of the access sheath is skived or trimmed to expose a portion of the metal braid. The electrical cable is joined at a joint to the exposed braid. Then, another outer layer or electrical insulation is applied to the joint to electrically isolate and strengthen the joint.

Additionally, the electrical conductor extending through the access sheath from the controller to the active electrode may vary. In embodiments, the configuration of the electrical conductor is a braid as described above. In another embodiment, the electrical conductor is a separate insulated cable extending through the tubular shaft and fixed in position. In another embodiment, the electrical conductor is a separate axially movable component (e.g. stylet) that passes through the lumen of the sheath and couples with the active electrode at the distal end. Indeed, the electrical conductor may have a wide variety of configurations.

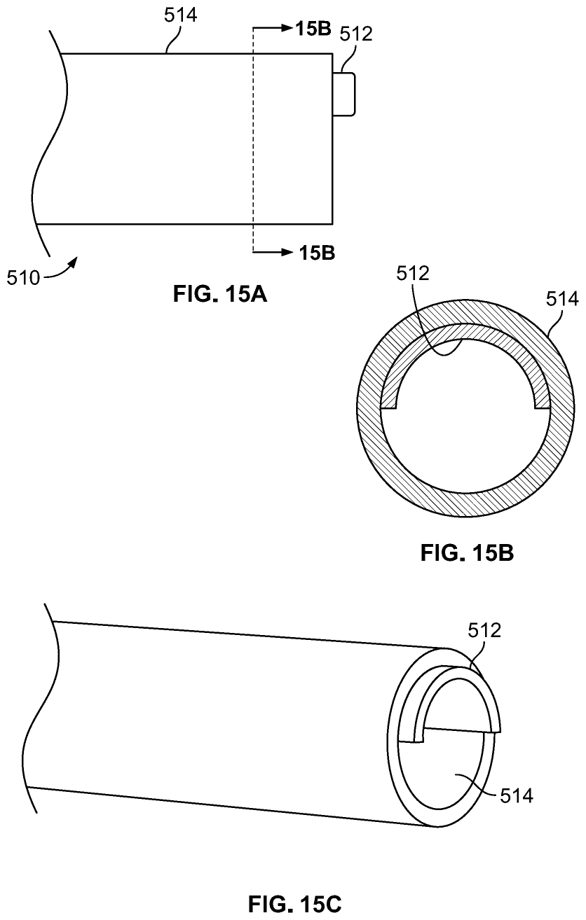

FIGS. 15A, 15B are partial side and cross sectional views respectively of another electrosurgical access sheath 510. The electrosurgical access sheath 510 is similar to that described above in connection with FIG. 9. Sheath includes a large distal active electrode 512 and a large tubular shaft 514 for tools to be advanced therethrough. However, the shape of the electrode is different than that shown in FIG. 9. The cross-sectional shape of the active electrode 512 shown in FIGS. 15A-15B is C-shaped. The materials and fabrication may otherwise be similar to that described herein.

FIG. 15C is a partial perspective view of the electrosurgical access sheath 510 shown in FIGS. 15A, 15B.

Although a number of specific shapes are described herein, the cross-sectional shape of the active electrode may vary widely. The shape may be circular or non-circular. The shape may protrude from the end of the access sheath and include only a front edge and little or no lateral surface for cutting. The electrode may take a form somewhat analogous to a cookie-cutter type stencil or die for cutting through substrates such as, e.g., thin airway walls. Plugs of different shapes corresponding to the cutter are carved including circles, gunsight, stars, bulls-eye, triangles, squares, and other geometries. The shapes may be formed as a group of discrete slits, or one contiguous pattern.

Additionally, electrode geometry corresponding to cross or perpendicular hatches may be incorporated into the basic shape. Cross hatches may be added to, e.g., a circle. Hatches may be perpendicular to the circle and radially oriented.

Non-limiting preferable characteristic cross dimensions for the die (e.g., diameter or length, or height) are in the range of a 1.5−3 mm.

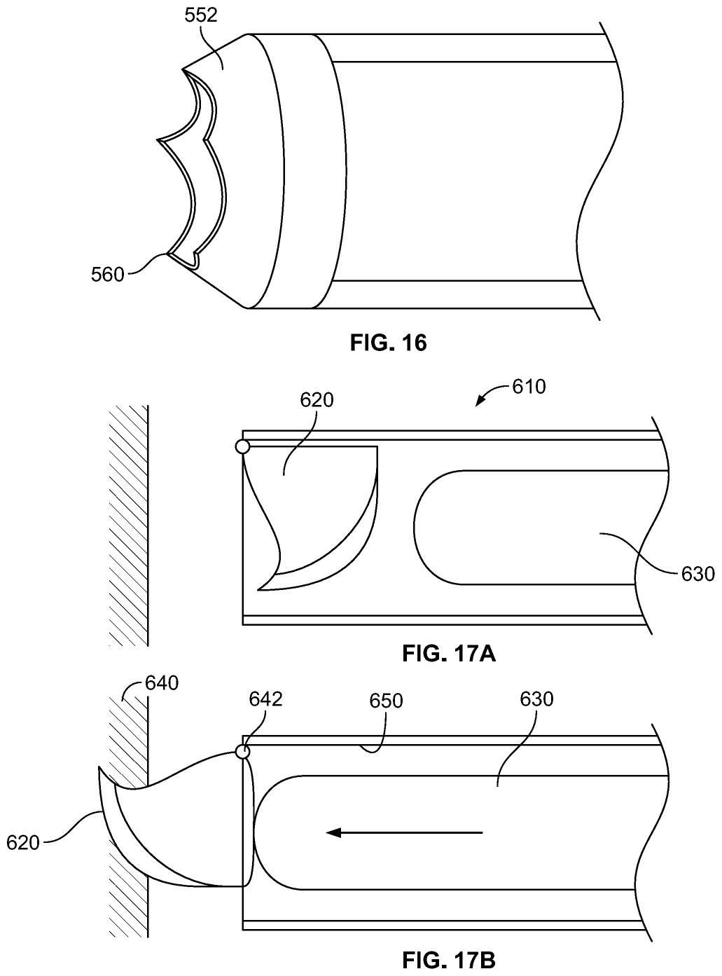

FIG. 16 shows an active electrode 552 having pointed or sharp features 562 to aid in cutting and to reduce the penetration force. The active electrode 552 in FIG. 16 includes tines 562.

FIGS. 17A, 17B are illustrations of another electrosurgical access sheath 610 having a cutting member 620 in a retracted and extended state respectively.

FIG. 17B shows the cutting member 620 urged by stylet pusher 630 into the tissue 640. The cutting member 620 is shown pivoting from a retracted position into an extended position about axis 642. Additionally, during actuation, pusher may deliver electrical waveforms to enable the cutting member to electrosurgically cut through the tissue 640. In alternative embodiments, the pusher configuration and cutting is purely mechanical.

Subsequent to cutting the hole through the tissue 640, the sheath 610 may be advanced towards the ROI (not shown). With the cutting member 642 fully rotated out of the lumen 650 (not shown), the pusher 630 may be exchanged with one or more ancillary tools to carry out an ancillary medical procedure on the ROI.

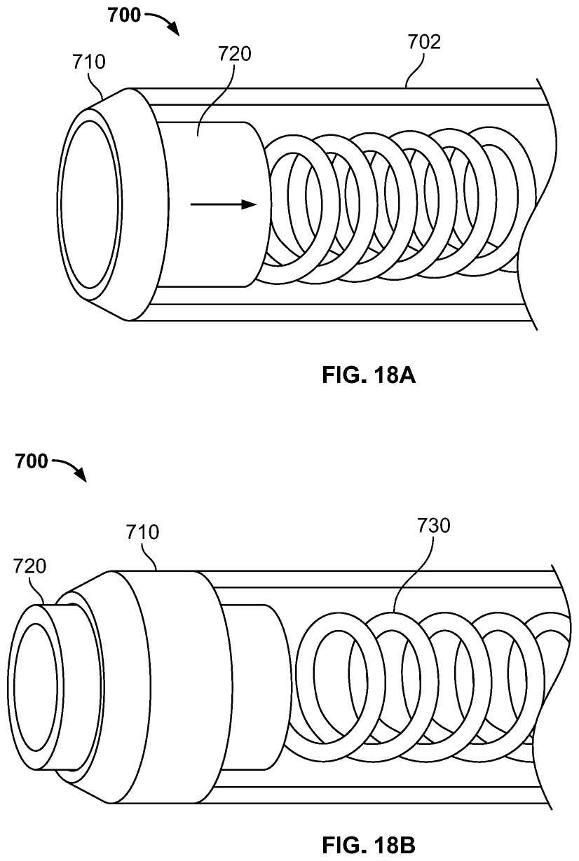

FIGS. 18A, 18B are partial illustrations of another electrosurgical access sheath 700 having another type of cutter 710 in an exposed and neutralized state respectively.

In FIG. 18A, the cutting member 710 is shown bare or exposed. It has an annular tip. The cutting member 710 may have, e.g., the shape of a coring needle.

A protective end 720 in the shape of a cylinder is shown retracted or withdrawn in elongate tube 702.

FIG. 18B illustrates the protective end 720 extended from the cutting tip 710, preventing or neutralizing the cutting action.

The actuation between the cutting tip 710 and protective cylinder 720 may be carried out by displacing one member or the other. The protective end 720, may be, for example, biased to extend beyond the cutting member via a spring 730. However, other actuating mechanisms may be employed including pull wires, and more sophisticated actuating elements.

Additionally, the cutting member and or protective cylinder may be utilized as an active electrode to facilitate cutting.

After the hole is created, ancillary tools may be advanced through the lumen of the shaft 702 as described herein.

Although a number of embodiments have been disclosed above, it is to be understood that other modifications and variations can be made to the disclosed embodiments without departing from the subject invention.

1. An electrosurgical access catheter for assisting a physician perform an ancillary surgical procedure on a region of interest (ROI) in a lung of a patient, the catheter comprising:
a tubular shaped elongate shaft, the shaft comprising a proximal section, a flexible distal section and a distal end;
a ring-shaped active electrode located at the distal end;
a passageway extending from the proximal section to the distal end, and through the ring-shaped active electrode; and
an electrical conductor extending from the proximal section to the distal section, and in electrical contact with the active electrode;
wherein the proximal section of the elongate shaft is detachably coupled to an electrosurgical controller such that the ring-shaped active electrode is operable with the electrosurgical controller and a dispersive electrode to electrosurgically make a circular-shaped incision through an airway wall, as the ring-shaped active electrode is advanced through the airway wall.

2. The electrosurgical access catheter of claim 1, wherein the dispersive electrode is a pad affixed to the skin of the patient.

3. The electrosurgical access catheter of claim 1, wherein the shaft comprises an outer polymer layer.

4. The electrosurgical access catheter of claim 1, wherein the electrical conductor is a braid coaxially arranged and extending from the proximal section to the distal end of the shaft.

5. The electrosurgical access catheter of claim 1, wherein the ring-shaped active electrode has a length less than or equal to 1 mm.

6. The electrosurgical access catheter of claim 1, wherein active electrode is made of a material visible under fluoroscopy.

7. The electrosurgical access catheter of claim 1, wherein the active electrode comprises a blunt distal edge.

8. The electrosurgical access catheter of claim 1, wherein the ring-shaped active electrode comprises a wall defining a tubular lumen and having an inner diameter of at least 2 mm.

9. The electrosurgical access catheter of claim 8, wherein the ring-shaped active electrode comprises an outer diameter of less than 3 mm.

10. The electrosurgical access catheter of claim 1, wherein the active electrode is operable to cut tissue when a sinusoidal voltage waveform from the electrosurgical controller is applied at a frequency between 300 kHz and 500 kHz.

11. The electrosurgical access catheter of claim 1, wherein the electrical conductor has a flexibility effecting form selected from the group consisting of variable braid pitch, transition from braid to a coil, and variable thickness.

12. The electrosurgical access catheter of claim 1, wherein the electrical conductor is axially movable and separable from the shaft.

13. The electrosurgical access catheter of claim 12, wherein the electrical conductor is a stylet.

14. An electrosurgical access catheter for assisting a physician perform an ancillary surgical procedure on a region of interest (ROI) in a lung of a patient, the catheter comprising:
a tubular shaped elongate shaft, the shaft comprising a proximal section, a flexible distal section and a distal end;
an active electrode located at the distal end, the active electrode comprising a die-shaped body, and an opening extending therethrough;
a passageway extending from the proximal section to the distal end, and through the active electrode; and
an electrical conductor extending from the proximal section to the distal section, and in electrical contact with the active electrode;
wherein the proximal section of the elongate shaft is detachably coupled to an electrosurgical controller such that the active electrode is operable with the electrosurgical controller and a dispersive electrode to electrosurgically make an incision through an airway wall corresponding to the shape of the die-shaped body as the active electrode is advanced through the airway wall.

15. The electrosurgical access catheter of claim 14, wherein the active electrode has a c-ring profile.

16. The electrosurgical access catheter of claim 14, wherein the active electrode has a cross sectional shape selected from the group comprising an annulus, bulls-eye, semi-circle, star, and gun-sight.

17. The electrosurgical access catheter of claim 14, wherein the active electrode has a length less than or equal to 1 mm.

18. The electrosurgical access catheter of claim 14, wherein the active electrode is made of a material visible under fluoroscopy.

19. The electrosurgical access catheter of claim 14, wherein the active electrode comprises a blunt distal edge.

20. An electrosurgical guide assembly for assisting a physician perform an ancillary surgical procedure on a region of interest (ROI) in a lung of a patient comprising:
an elongate guide member, the guide member comprising a proximal section, an intermediate section, and a distal section, and an active electrode located in the distal section;
an electrical conductor extending from the proximal section to the distal section of the guide member, and in electrical contact with the active electrode;
an elongate flexible access sheath coaxially advanceable over the elongate guide member; and
wherein the proximal section of the elongate guide member is detachably coupled to an electrosurgical controller such that the active electrode is operable with the electrosurgical controller and a dispersive electrode to electrosurgically make an incision through an airway wall as the active electrode is activated and advanced through the airway wall.

21.-54. (canceled)
